(19) United States
(12) Patent Application Publication
(10) Pub. No.: US 2003/0144586 A1
(43) Pub. Date: Jul. 31, 2003

(54) Biological information sensing device

(76) Inventor: Keisuke Tsubata, Chiba-shi (JP)

Correspondence Address: ADAMS & WILKS, 31st Floor, 50 Broadway, New York, NY, 10004, US

(21) Appl. No.: 10/349,884

(22) Filed: Jan. 23, 2003

(30) Foreign Application Priority Data
Jan. 31, 2002 (JP) 2002-022822

(51) Int. Cl. A61B 5/05

(52) U.S. Cl. 600/407

(57) ABSTRACT

A biological information sensing device has a sensor portion formed in an elongate strap that can be attached to a living organism such that the sensor portion is maintained in intimate contact with a measurement area of the living organism. A plurality of signal processing portions are also formed in the elongate strip for processing biological information, such as pulse rate, blood pressure or serum concentrations, sensed by the sensor portion.

### BACKGROUND OF THE INVENTION

The present invention relates to a biological information sensing device for sensing biological information such as arterial pulses or the like, and more particularly, to a biological information sensing device of a type like an arterial pulse wave detector which is wrapped around the wrist or the like.

A portable arterial pulse wave detector has been proposed which is provided with a pressure forming elastic piece at an intermediate portion of a strap such that the elastic piece may press a sensor portion against an area of the wrist surface near the radial artery of the wrist for holding the sensor portion in intimate contact with the wrist surface area (JP-A-8-52118).

However, the proposed portable arterial pulse wave detector has a drawback that precise measurements of arterial pulses cannot be obtained. This is because when its main body (head portion) unifying a display panel and processing circuit of great masses is subjected to a relatively great accelerative motion associated with the motion of the wrist during exercise or the like, for example, an inadvertent inertia force apt to rotate/displace the head portion about the wrist is applied to the head portion, thus varying the pressure on the sensor portion connected with the head portion for pressing the sensor portion against the wrist surface or causing the rotation/displacement of the sensor portion.

In view of the foregoing, it is an object of the invention to provide a biological information sensing device which is capable of minimizing the effect of the inertia force on the device thereby minimizing the displacement thereof, irrespective of exposure to the accelerative motion during training or other exercises.

### SUMMARY OF THE INVENTION

In accordance with the invention for achieving the above object, a biological information sensing device applied to a neck-like portion, inclusive of a predetermined measurement area, of a living organism as holding a sensor portion thereof in intimate contact with the measurement area for sensing biological information, the sensing device is characterized in that a variety of signal processing portions for handling the biological information sensed by the sensor portion take a form of an elongate strap as a whole in order to be wrapped around an outer periphery of the neck-like portion along with the sensor portion. The elongate strap is defined as follows. Assuming that, for example, a section of the wrist is an oval shape consisting of two opposite curved lines having small curvatures and two opposite curved lines having great curvatures, the strap has such a length as to cover at least two apexes of the curved lines.

In the biological information sensing device according to the invention, the variety of signal processing portions for handling the biological information sensed by the sensor portion take the form of an elongate strap as a whole such that the signal processing portions along with the sensor portion may be wrapped around the neck-like portion thereby uniformly distributing the masses along an extension direction of the strap. Therefore, the inertia force applied to the strap in conjunction with a sudden accelerative or decelerative motion can be uniformly distributed and hence, there occurs little force acting to rotate the strap. As a result, the displacement of the sensor is minimized.

The signal processing portions and the sensor portion constituting the main body of the sensing device are formed on a flexible substrate, for example, in a continuous fashion. However, at least one of the signal processing portions or the processing portions may be formed on a rigid substrate or on individual rigid substrates while the rigid substrate(s) may be connected via a flexible cable or the like. In either cases, it is preferred that the signal processing portions constituting the elongate strap-like main body of the sensing device present a uniform mass distribution along the longitudinal direction of the strap, with the proviso that the respective functions of the signal processing portions are not decreased. In other words, for the purpose of uniformly distributing the masses, the location of the circuit components may be properly changed as required, or otherwise, a kind of weights or masses having no effect on the functions of the main body of the sensing device may be attached to desired places along the longitudinal direction of the strap-like main body so long as such weights or masses do not results in an excessive increase of the mass of the main body of the sensing device.

It is noted here that the strap-shaped main body of the biological information sensing device, which includes the sensor portion and the variety of signal processing portions, may be provided with a strap fastening hardware at an end thereof with respect to the longitudinal direction thereof. Furthermore, the biological information sensing device may further comprise, in addition to the strap-shaped main body of the sensing device including the sensor portion and the signal processing portions, strap-like fastening means for wrapping and fastening the main body of the sensing device around the neck-like portion.

In the former case, the sensing device features an easy handling because the main body thereof is integrated with the strap fastening hardware. In the latter case, the main body of the sensing device is adapted for positional adjustment (positional change) relative to the strap-like fastening means and hence, the main body of the sensing device with the sensor portion precisely positioned at the measurement area can be stably secured to place by means of the strap-like fastening means, irrespective of the size or the like of the neck-like portion.

In another aspect of the invention for achieving the above object, a biological information sensing device applied to a neck-like portion, inclusive of a predetermined measurement area, of a living organism as holding a sensor portion thereof in intimate contact with the measurement area for sensing biological information, the sensing device comprises a signal processing portion for processing the biological information sensed by the sensor portion, and a display/transmission portion for displaying/transmitting the processed signal, wherein the signal processing portion and the display/transmission portion take a form of an elongate strap as a whole in order to be wrapped around the neck-like portion along with the sensor portion.

In this case, as well, the strap-shaped main body of the sensing device including the signal processing portion and the display/transmission portion may be provided with a strap fastening hardware at an end thereof with respect to the longitudinal direction thereof. Further, the biological information sensing device may further comprise, in addition to the strap-shaped main body of the sensing device including the sensor portion, the signal processing portion and the display/transmission portion, strap-like fastening means for wrapping and fastening the main body of the sensing device around the neck-like portion. Both of the cases have the same features as those described above.

Although the neck-like portion is typically the wrist portion for example, other areas such as the cervical region with the carotid artery may be included.

The biological information to be sensed typically includes for example information related to arterial pulses such as frequency of pulse and the like (accordingly, the biological information sensing device is a so-called arterial pulse wave detector). However, the biological information sensing device may also handle other biological information or signals indicative of blood pressure, serum concentrations of a particular component in blood and the like, or indicative of body fluids other than blood.

The biological information sensing device according to the invention permits the sensor portion to be stably held against or secured to the measurement area of the living organism so that the biological information (frequency of pulse, variations of blood pressure, level of oxygen in blood and the like) during exercise can be sensed precisely.

In a case where the biological information sensing device comprises an arterial pulse wave detector including an arterial pulse wave sensor based on supersonic wave, the biological information sensing device comprises, for example, an arterial pulse wave sensor portion including a supersonic transmitter and a supersonic receiver; and besides, a variety of signal processing portions which include an oscillating/actuating portion for the supersonic transmitter of the sensor portion, an arterial pulse wave receiver portion for extracting an analog arterial pulse signal from a supersonic signal received by the supersonic receiver of the sensor portion, a digital signal processing portion for converting the arterial pulse signal extracted by the arterial pulse wave receiver into a digital signal and processing the resultant digital signal, and a display portion for display of the results of the signal processing done by the digital signal processing portion. These signal processing portions are individually formed on separate rigid or flexible circuit boards to form separate blocks which are connected into a strap form by means of a flexible cable or the like. As mentioned supra, however, at least one of the signal processing portions may be divided into separate blocks or at least some of the signal processing portions may be combined into one block in order that the masses can be uniformly distributed. Incidentally, a power source such as a battery relatively prone to heavy weight may be located at a suitable place for accomplishing the uniform mass distribution of the strap. If required, the power source may consist of a plurality of batteries which may be distributed along the length of the strap-like main body of the sensing device.

### BRIEF DESCRIPTION OF THE DRAWINGS

FIG. 1 are a group of diagrams showing an arterial pulse wave detector according to one preferred embodiment of the invention, FIG. 1A representing a perspective view explanatory of a main body of the arterial pulse wave detector of one preferred embodiment, FIG. 1B representing a sectional view explanatory of a state where the arterial pulse wave detector of one preferred embodiment including the main body of FIG. 1A is worn on the wrist, FIG. 1C representing a sectional view resemblent to FIG. 1B and explanatory of an exemplary modification of FIG. 1B, and FIG. 1D representing a sectional view resemblent to FIG. 1B and explanatory of another exemplary modification of FIG. 1B;

FIG. 2 are a group of block diagrams illustrating the functions of the arterial pulse wave detector shown in FIGS. 1A and 1B, FIG. 2A representing a functional block diagram showing an example of the arterial pulse wave detector shown in FIGS. 1A and 1B, and FIG. 2B representing a functional block diagram showing an exemplary modification of FIG. 2A;

FIG. 3 are a group of schematic time charts of signals processed by the arterial pulse wave detector shown in FIGS. 1 and 2, FIG. 3A representing a transmitted supersonic signal, FIG. 3B representing a received supersonic signal modulated in frequency by the Doppler effect, FIG. 3C representing an amplitude modulated signal obtained by differential amplification, and FIG. 3D representing a signal of an extracted amplitude component;

FIG. 4 is a perspective view explanatory of a sensor portion employed by the arterial pulse wave detector shown in FIGS. 1 and 2; and

FIG. 5 are a group of diagrams showing other preferred embodiments of the arterial pulse wave detector of the invention, FIG. 5A representing a sectional view explanatory of a state where one preferred embodiment of the arterial pulse wave detector including the main body of FIG. 1C is worn on the wrist, FIG. 5B representing a sectional view resemblent to FIG. 5A and explanatory of a state where an arterial pulse wave detector including the main body of FIG. 1D is worn on the wrist, and FIG. 5C representing a sectional view resemblent to FIG. 5A and explanatory of a state where an arterial pulse wave detector including a modification of the main body is worn on the wrist.

### DESCRIPTION OF THE PREFERRED EMBODIMENTS

Next, some of the preferred modes of the invention will be described with reference to the preferred embodiments thereof shown in the accompanying drawings.

### EXAMPLES

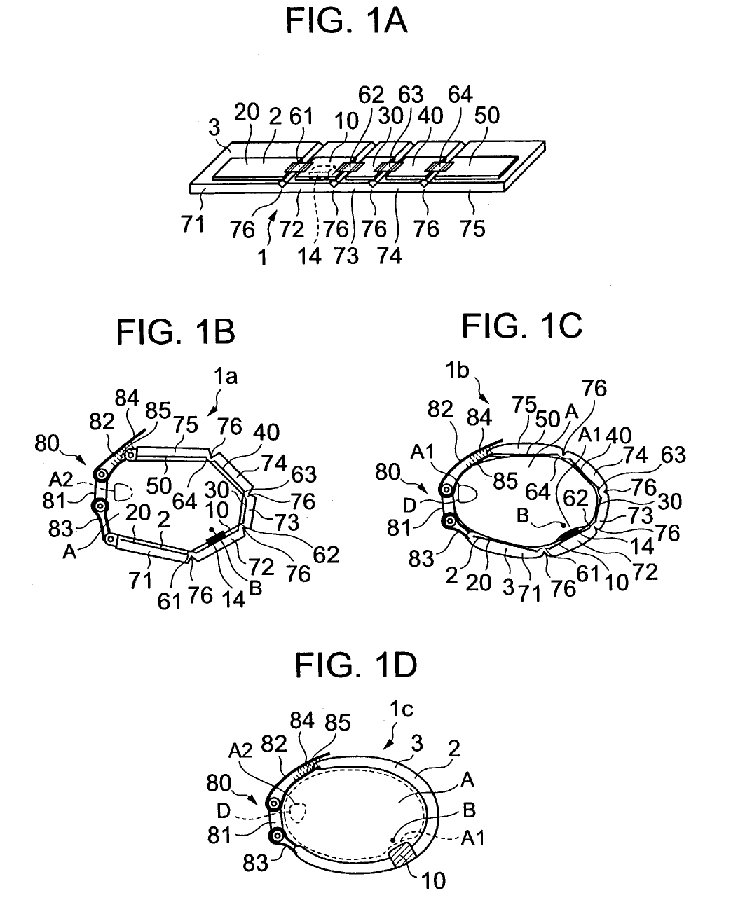

Out of FIGS. 1 to 4 for illustrating an arterial pulse wave detector 1 as a biological information sensing device according to one preferred embodiment of the invention, FIG. 1 are a general view showing the arterial pulse wave detector 1.

As shown in FIG. 1A for example, the arterial pulse wave detector 1 includes a strap-like main body 2 of the detector, and a strap-like support portion 3 for supporting the main body 2 of the detector.

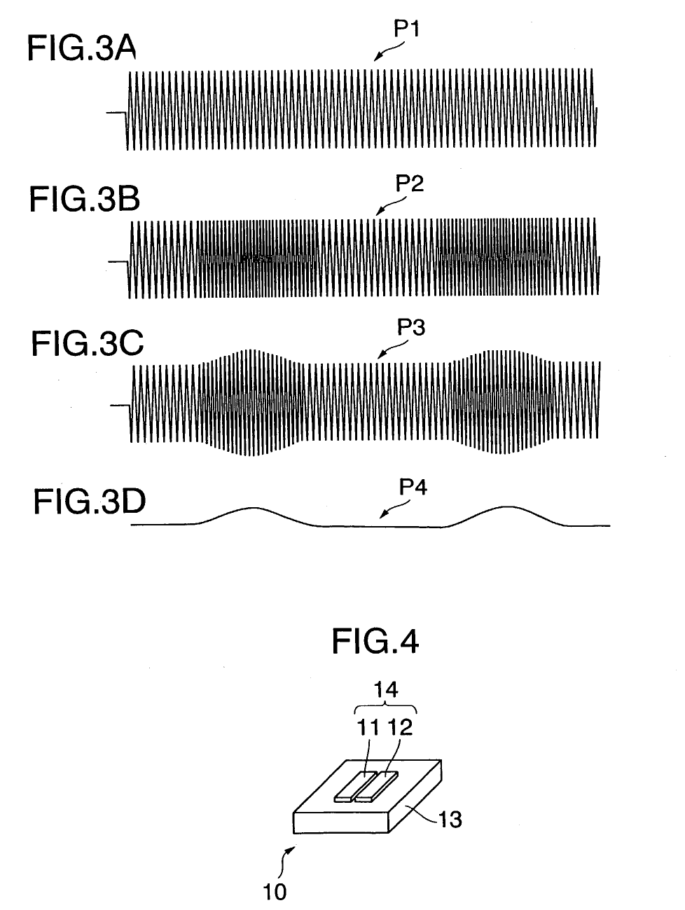

The main body 2 of the detector includes an arterial pulse wave sensor portion 10 including a supersonic signal transmitter/receiver portion or sensor body 14 comprising a supersonic transmitter 11 and a supersonic receiver 12; and besides, a variety of signal processing portions 20, 30, 40 and 50 which include an oscillating/actuating portion 20 for the supersonic transmitter 11 of the sensor portion 10, an arterial pulse wave receiving portion 30 for extracting an analog arterial pulse signal P4 from a supersonic signal P2 (FIG. 3) received by the supersonic receiver 12 of the sensor portion 10, a digital signal processing portion 40 for converting the arterial pulse signal P4 extracted by the arterial pulse wave receiving portion 30 into a digital signal and processing the resultant digital signal, and a display portion 50 for displaying the results of the signal processing done by the digital signal processing portion 40.

The circuit components 10, 20, 30, 40 and 50 constituting the main body 2 of the detector each comprise, for example, a circuit board and a circuit device incorporated in the circuit board. The circuit components 20, 10, 30, 40 are each connected with the respective adjoining circuit components 10, 30, 40 and 50 via respective flexible cables 61, 62, 63 and 64. It is noted here that each of the circuit boards may be a printed wiring board such as formed of a resin or ceramic, or a circuit board perse forming an integrated circuit board. In the example shown in FIG. 1A, the circuit board itself typically has rigidity but may have flexibility. Additionally, the circuit components 20, 30, 40, 50 may be accommodated in a case such as a plastic case, respectively.

The strap-like support portion 3 is formed of a flexible strap material such as a urethane resin, and includes, for example, circuit components 20, 10, 30, 40 and 50,individually serving to support their respective strap forming bases 71, 72, 73, 74 and 75 and interconnection portions 76 for interconnecting the forming bases 71 to 75. The strap forming bases 71, 72, 73, 74 and 75 of the strap-like support portion 3 have the corresponding circuit components 10, 20, 30, 40 and 50 laid thereon or embedded therein. In this connection, the strap forming bases 71, 72, 73, 74 and 75 are previously formed with recesses or openings on either one surface or both surfaces thereof for receiving therein the corresponding circuit components 20, 10, 30, 40 and 50. The circuit components 20, 10, 30, 40 and 50 are fixed in the recessesor openings by disposing, engagement or bonding. In this case, the circuit components 20, 10, 30, 40 and 50 may be individually disposed in the corresponding recesses while adjoining circuit components may be interconnected by means of the respective flexible cables 61, 62, 63 and 64. If desired, of course, at least some or all of the strap forming bases 71, 72, 73, 74 and 75 may be formed integrally with the corresponding circuit components 20, 10, 30, 40 and 50 so as to at the forming of the strap forming bases 71, 72, 73, 74 and 75 be embedded in the corresponding strap forming bases 71, 72, 73, 74 and 75 of the strap-like support portion 3.

More specifically, the strap-like support portion 3 is provided with a strap fastening structure 80 on opposite ends thereof, as shown in FIG. 1B or 1C, the strap fastening structure 80 comprising a strap engaging hardware 81 as a strap fastening portion, and a fastening strap piece 82. The strap fastening structure 80 may be formed in a desired fashion according to a material used or fastening strength.

FIG. 1B shows an arterial pulse wave detector 1a wherein the elements 71-75 of the strap-like support portion 3 are formed of a material having a relatively high rigidity and the interconnection portions 76 are flexible, whereas FIG. 1C shows an arterial pulse wave detector 1b wherein the elements 71-75 of the strap-like support portion 3 are formed of a soft material having a relatively high flexibility so that the interconnection portions 76 as well as the elements 71-75 are flexible and round. On the other hand, FIG. 1D shows an arterial pulse wave detector 1c wherein the circuit boards themselves are also flexible and incorporated into the strap-like support portion 3 for distributing the masses as uniformly as possible. Thus, the elements 20, 30, 40 and 50 of the main body 2 of the detector, but for the sensor portion 10 including the transmitter/receiver portion 14, practically belong to the flexible strap-like support portion 3.

As shown in FIG. 4 for example, the sensor portion 10 comprises a common substrate 13 incorporating therein the supersonic transmitter/receiver or the sensor body 14 which includes the supersonic transmitter 11 and the supersonic receiver 12 individually including a piezoelectric device. As seen in FIGS. 1B-1D, the supersonic transmitter 11 and receiver 12 of the sensor portion 10 are placed in a manner to be properly pressed into intimate contact with a wrist surface area A1 where the radial artery B through the human wrist A is closest to the wrist surface. More specifically, in order to accomplish an effective fastening/fixing of the strap-like support portion 3 when the supersonic transmitter/receiver portion 14 of the sensor portion 10 is placed on the wrist surface area A1 close to the radial artery B, the strap-like support portion 3 is adjusted lengthwise so as to position the strap fastening structure 80 at a bumped side A2 of the wrist A near the cubitus D. In order to permit such a positional adjustment, a strap portion 83 supporting the hardware 81 may be adapted for lengthwise adjustment.

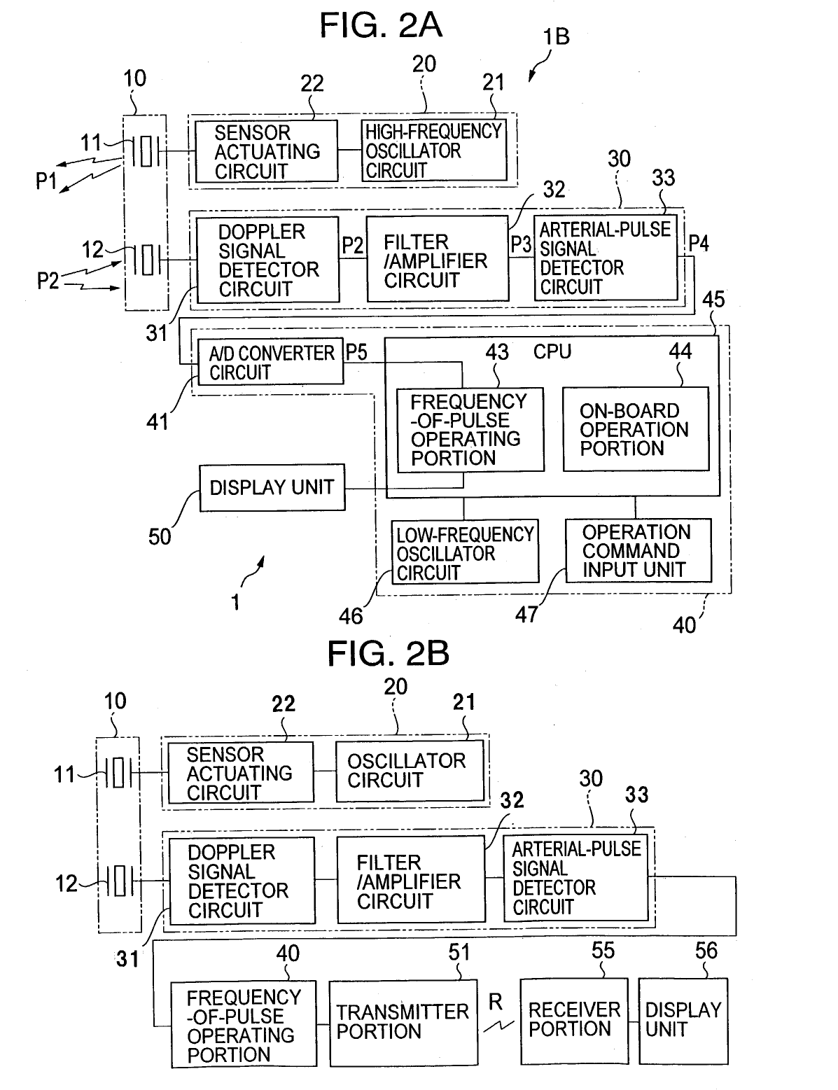

In the arterial pulse wave detector 1, as shown in FIG. 2A, the supersonic transmitter 11 of the sensor portion 10 is actuated to transmit a supersonic signal P1 under the control of the oscillating/actuating circuit portion 20 including a high-frequency oscillator circuit 21 and a sensor actuating circuit 22 while the signal P1 is reflected as impinging upon blood components, such as blood cells or the like, in blood flowing through the radial artery B. The supersonic signal emitted from the supersonic transmitter 11 is typically the signal P1 practically having a constant frequency and amplitude, as shown in FIG. 3A for example.

A supersonic signal P2 reflected by the blood components in blood as a pulsing stream through the radial artery B and received by the supersonic receiver 12 is modulated in frequency due to the Doppler effect associated with the pulse of the blood components as the reflector of the transmitted supersonic signal P1. Thus, the signal P2 assumes a form as shown in FIG. 3B, for example.

As shown in FIG. 2A for example, the arterial pulse wave receiving portion 30 for extracting the analog arterial pulse signal P4 from the supersonic signal P2 received by the supersonic receiver 12 of the sensor portion 10 includes a doppler signal detector circuit 31, a filter/amplifier circuit 32 and an arterial pulse signal detector circuit 33. An output from the doppler signal detector circuit 31 is, for example, an electrical signal of a wave form P2 similar to that of the received supersonic signal P2. The filter/amplifier circuit 32 amplifies an amount of variation of the doppler signal P2˜sin{(ω+Δω)t} using the original transmission signal P1˜sin(ω·t) as a reference or a reference signal, so as to extract a differential amplification signal P3˜{sin(Δω/2)t}·sin{ω−(Δω/2)}t as shown in FIG. 3C. It is noted here that ω denotes an angular frequency of the supersonic signal P1, and that Δω=Δω(t) denotes a modulated angular frequency dependent upon time t due to the Doppler effect. In the arterial pulse wave receiving portion 30, the arterial pulse signal detector circuit 33 extracts, as the arterial pulse signal P4, an amplitude modulated component sin(Δω/2)t from the differential amplification signal P3. In the case of a square law detection, the arterial pulse component can be extracted as sin(Δω)t.

Although FIG. 3 show the arterial pulse wave P4 quite in a simple wave form, the arterial pulse wave P4 actually presents much more complicated time-dependent wave form than that of FIG. 3D. Particularly in a state where the cardio pulmonary circulatory system is over taxed during or after exercise, the arterial pulse wave P4 assumes a much more complicated and irregular wave form containing a wide range of high frequency components.

In the case of an arterial pulse wave detector 1 shown in FIG. 2A, the digital signal processing portion 40 includes an analog/digital (A/D) converter circuit 41 for converting the analog signal P4 indicative of the arterial pulse wave into a digital signal P5 indicative of the arterial pulse wave; a central processing unit (CPU) 45 for receiving the digital arterial pulse signal P5; and a low-frequency oscillator circuit 46 for supplying the CPU 45 with a reference signal for processing. In this case, the CPU 45 includes a memory for storing a frequency-of-pulse operation program and a microprocessor for executing the program, thus forming a frequency-of-pulse operating portion 43 for operating the frequency of pulse based on the digital arterial pulse signal P5 with reference to the low-frequency signal from the low-frequency oscillator circuit 46. Typically, the CPU forms a digital signal processor (DSP) wherein a part of the frequency-of-pulse operation program including a fast Fourier transformation (FFT) process is incorporated in a digital signal processor circuit. It is noted that the CPU 45 further includes a device operation portion 44 for receiving an operation command from an operation command input portion 47 such as a push-button switch.

According to FIG. 2A, the display portion 50 comprises a display unit such as a liquid crystal panel for displaying the operation result or frequency of pulse Q determined by the frequency-of-pulse operating portion 43 of the CPU 45.

According to the foregoing description, the A/D converter circuit 41, the operation command input portion 47 and the like belong to the digital signal processing portion 40. However, the A/D converter circuit 41 may belong to, for example, an output circuit portion of the arterial pulse wave receiving portion 30 for processing the analog signal. Further, the operation command input portion 47 like a push-button switch may be integrally formed with the display unit 50 as an article. Similarly, the other components may be freely combined into blocks, as desired, so long as such combinations contribute to the mass distribution as a whole.

Needless to say, the CPU 45 may perform other operations during spare-time when the frequency of pulse Q is not operated or at an interval between the operations of the frequency of pulse Q. One example of the other operations include a time counting operation as a clock. Specifically, the CPU 45 is, for example, capable of performing the time counting operation as a timer and hence, the display portion 50 is also capable of functioning as a display of a digital clock.

The arterial pulse wave detector 1 (more specifically, the detector 1a, 1b and 1c which are represented by the reference numeral 1 in the this paragraph) of the above construction may be worn on the wrist A as follows. The arterial pulse wave detector 1 is placed around the wrist A in a manner to bring the sensor body 14 of the sensor portion 10 into abutment against the wrist surface area A1 near the radial artery B of the wrist A. With the hardware 81 positioned at the wrist bump area A2 near the cubitus D, the strap piece 82 is threaded through the hardware 81 and then fixed by means of engagement portions 84, 84 such as hook and loop fasteners.

When the arterial pulse wave detector 1 is wrapped around the wrist A in this manner, the main body 2 and strap-like support portion 3 of the arterial pulse wave detector 1 have substantially uniform mass distribution along the longitudinal direction thereof. Therefore, even when the wrist A is subjected to the accelerative motion due to exercise or the like, such a great inertia force as to bring the arterial pulse wave detector 1 into mono-directional rotation about the wrist A will not actually occur. Accordingly, the sensor body 14 of the sensor portion 10 of the arterial pulse wave detector 1 is maintained in intimate contact with the measurement area A1, thus achieving the precise measurement of the arterial pulses.

Another approach may replace the direct display of the frequency of pulse Q on the display portion 50 shown in FIG. 2A. As shown in FIG. 2B, a transmitter portion 50 including an antenna or coil is adapted to transmit the data on the frequency of pulse Q, obtained by the frequency-of-pulse operating portion 40, in the form of an electromagnetic signal R such as of an electromagnetic wave or variable magnetic field, whereas a separate receiver portion 55 receives the electromagnetic signal R, from which the frequency of pulse Q is extracted to be displayed on a display unit 56. In this case, an arterial pulse wave detector 1B worn on the wrist of one arm may take the form of a headless strap as shown in FIGS. 1B-1D or a strap free from mass concentration thus presenting wide mass distribution whereas the frequency of pulse Q may be displayed on the display unit 56 with a clock function which is worn on the other arm, for example. This permits the whole body of the display unit 56 prone to heavy weight to be mechanically separated from the arterial pulse wave detector 1B, contributing to the weight reduction and mass distribution of the arterial pulse wave detector 1B. As a result, the arterial pulse wave detector 1B is less susceptible to such a force as to move the sensor body 14 of the arterial pulse wave detector 1B relative to the wrist A or such a force as to cause the variations of the pressure for pressing the sensor body 14 against the wrist A. Thus, the arterial pulse wave detector 1B can more precisely or more reliably take measurements.

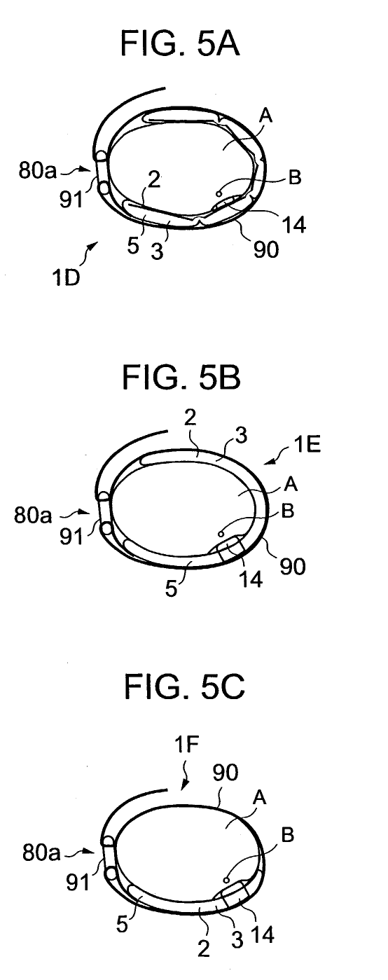

The arterial pulse wave detector wherein the strap fastening structure 80 is directly attached to an end of the strap-like support portion 3 with the main body 2 of the detector incorporated therein may have an alternative mode of an arterial pulse wave detector 1D. As shown in FIG. 5A, the arterial pulse wave detector 1D has an arrangement wherein a strap-like measuring unit 5 formed by incorporating the strap-like main body 2 of detector in the strap-like support portion 3 is adapted to be fastened to a predetermined position of the wrist A by means of a measuring-unit fastening strap 90 provided with a strap fastening structure 80a resemblent to the strap fastening structure 80 at an end thereof. In the arterial pulse wave detector 1D, the strap-like measuring unit 5 and the fastening strap 90 are formed as independent articles. The strap-like measuring unit 5 is adapted for positional adjustment relative to the fastening strap 90. Accordingly, in a state where the sensor body 14 of the sensor portion 10 of the detector body 2 of the strap-like measuring unit 5 is exactly positioned at the optimum place A1 corresponding to the radial artery B, a strap fastening hardware 91 of the fastening structure 80a of the fastening strap 90 can be positioned at the bump place A2 near the cubitus D where the fastening structure is optimally secured to the wrist A. If desired in this case, the unit 5 may be locked to the fastening strap 90 by means of engaging or locking means such as hook and loop fasteners in a manner to be adapted for positional adjustment.

It is noted that FIG. 5A illustrates the arterial pulse wave detector 1D wherein the flexible strap-like portion 3 resemblent to that of FIG. 1C is adapted for application using the separate fastening strap 90, whereas FIG. 5B illustrates an arterial pulse wave detector 1E wherein the unit 5 comprising the flexible strap-like portion 3 and detector body 2 resemblent to those of FIG. 1D is adapted for application using the separate fastening strap 90. In a case where the unit 5 comprising the main body 2 of detector and the strap-like portion 3 is adapted for application using the separate fastening strap 90, an arterial pulse wave detector 1F may also be employed wherein, for example, shown in FIG. 5C, the unit 5 comprising the strap-like portion 3 and the main body 2 of detector extends along a part of an outside circumference of the wrist A (say, about a half or less of the outside circumference) rather than the substantially overall length of the outside circumference of the wrist.

In a case where the fastening strap 90 independent from the main body 2 of detector is used, the arterial pulse wave detector may dispense with the strap-like support portion 3 and have an arrangement wherein the main body 2 of detector extended in a strap form is directly fastened to the wrist by means of the fastening strap 90.

What is claimed is:

1. A biological information sensing device comprising:
a sensor portion held in intimate contact with a neck-like portion, inclusive of a predetermined measurement area, for sensing biological information; and
a plurality of signal processing portions for handling the biological information sensed by the sensor portion; wherein the signal processing portions take a form of an elongate strap as a whole in order to be wrapped around an outer periphery of the neck-like portion along with the sensor portion.

2. A biological information sensing device according to claim 1, wherein a strap-shaped main body of the biological information sensing device, which includes the sensor portion and the variety of signal processing portions, is provided with a strap fastening hardware at an end thereof with respect to a longitudinal direction thereof.

3. A biological information sensing device according to claim 1, further comprising, in addition to a strap-shaped main body of the biological information sensing device including the sensor portion and a variety of signal processing portions, strap-like fastening means for wrapping and fastening the main body of the biological information sensing device around the neck-like portion.

4. A biological information sensing device according to claim 1, wherein the neck-like portion is the wrist.

5. A biological information sensing device according to claim 1, wherein the neck-like portion is the cervical region.

6. A biological information sensing device according to claim 1, wherein the biological information includes at least one information item selected from the group consisting of the frequency of pulse, blood pressure and serum concentrations of a particular component.

7. A biological information sensing device comprising:
a sensor portion held in intimate contact with a neck-like portion, inclusive of a predetermined measurement area, for sensing biological information;
a signal processing portion for processing the biological information sensed by the sensor portion, and
a display/transmission portion for displaying/transmitting the processed signal,
wherein the signal processing portion and the display/transmission portion take a form of an elongate strap as a whole in order to be wrapped around the neck-like portion along with the sensor portion.

8. A biological information sensing device as claimed in claim 7, wherein a strap-shaped main body of the biological information sensing device including the sensor portion, signal processing portion and display/transmission portion is provided with a strap fastening hardware at an end thereof with respect to a longitudinal direction thereof.

9. A biological information sensing device as claimed in claim 7, further comprising, in addition to a strap-shaped main body of the biological information sensing device including the sensor portion, signal processing portion and display/transmission portion, strap-like fastening means for fastening the main body of the biological information sensing device around the neck-like portion.

10. A biological information sensing device according to claim 7, wherein the neck-like portion is the wrist

11. A biological information sensing device according to claim 7, wherein the neck-like portion is the cervical region.

12. A biological information sensing device according to claim 7, wherein the biological information includes at least one information item selected from the group consisting of the frequency of pulse, blood pressure and serum concentrations of a particular component.
